(19) United States
(12) Patent Application Publication
(10) Pub. No.: US 2023/0094891 A1
(43) Pub. Date: Mar. 30, 2023

(54) STIFF BRAID MEMBER FOR PROSTHETIC VALVE DELIVERY APPARATUS

(71) Applicant: Edwards Lifesciences Corporation, Irvine, CA (US)

(72) Inventors: Rupesh Gajanan Nawalakhe, Tustin, CA (US); Sandip Vasant Pawar, Irvine, CA (US)

(21) Appl. No.: 18/076,106

(22) Filed: Dec. 6, 2022

Related U.S. Application Data
(63) Continuation of application No. PCT/US2021/036192, filed on Jun. 7, 2021.
(60) Provisional application No. 63/037,779, filed on Jun. 11, 2020.

(51) Int. Cl. A61F 2/24

(52) U.S. Cl. CPC A61F 2/2466; A61F 2250/0017

(57) ABSTRACT

A braided member can include a first set of yarns extending in a first direction, a second set of yarns extending in a second direction and intertwined with the first set of yarns, and a set of axial yarns extending along a longitudinal axis of the braided member and disposed between the first set of yarns and the second set of yarns. The braided member can include a tubular braid.

### CROSS REFERENCE TO RELATED APPLICATION

This application is a continuation of PCT Application No. PCT/US2021/036192, entitled STIFF BRAID MEMBER FOR PROSTHETIC VALVE DELIVERY APPARATUS, filed Jun. 7, 2021, which claims the benefit of U.S. Provisional Application Ser. No. 63/037,779, entitled STIFF BRAID MEMBER FOR PROSTHETIC VALVE DELIVERY APPARATUS, filed on Jun. 11, 2020, where each of above-referenced is incorporated by reference herein in its entirety.

### FIELD

The present disclosure relates to implantable prosthetic devices, such as prosthetic heart valves, and to delivery apparatus and methods for implanting prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (e.g., stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient's vasculature (e.g., through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

Given the relatively high number of small components included in a typical delivery apparatus, it can be difficult and/or time-consuming for an assembler to assemble the delivery apparatus. Accordingly, there is a need for improved delivery apparatus, methods for assembling delivery apparatus, and methods for implanting prosthetic heart valves.

### SUMMARY

Described herein are prosthetic heart valves, embodiments of a delivery apparatus, and methods for implanting prosthetic heart valves.

In a representative embodiment, a braided member can comprise a first set of yarns extending in a first direction, a second set of yarns extending in a second direction and intertwined with the first set of yarns, and a set of axial yarns extending along a longitudinal axis of the braided member and disposed between the first set of yarns and the second set of yarns. The braided member can comprise a tubular braid.

In another representative embodiment, a braided member can comprise a first set of yarns extending in a first direction, a second set of yarns extending in a second direction and intertwined with the first set of yarns, and a set of axial yarns. The axial yarns can extend along a longitudinal axis of the braided member and can be disposed between the first set of yarns and the second set of yarns. The braided member can have a braid density between 10 and 400 PPI.

In another representative embodiment, a braided member can comprise an outer layer and an inner core member. The outer layer can comprise a first set of yarns extending in a first direction, a second set of yarns extending in a second direction and intertwined with the first set of yarns, and a set of axial yarns extending along a longitudinal axis of the braided member and disposed between the first set of yarns and the second set of yarns.

In a representative embodiment, a force balancing assembly can comprise two or more actuation members each comprising a cap member, at least one pulley member, and at least one braided member. The at least one braided member having a first end portion coupled to a first cap member, a second end portion coupled to a second cap member, and a body portion disposed around the at least one pulley member. The braided member comprising a first set of yarns extending in a first direction, a second set of yarns extending in a second direction and intertwined with the first set of yarns, and a set of axial yarns extending along a longitudinal axis of the braided member and disposed between the first set of yarns and the second set of yarns. The force balancing assembly can be configured to equally distribute a force between the two or more actuation members, and the braided member can have a braid density between 10 and 400 PPI.

The foregoing and other objects, features, and advantages of the invention will become more apparent from the following detailed description, which proceeds with reference to the accompanying figures.

### BRIEF DESCRIPTION OF THE DRAWINGS

FIG. 1 is a side elevation view of an exemplary embodiment of a delivery apparatus for a prosthetic heart valve.

FIG. 2 is a side elevation view of a portion of an exemplary embodiment of a braid.

FIG. 3 is a simplified view of a portion of the braid shown in FIG. 2, with the braid shown in a flattened configuration.

FIGS. 4A-7B illustrate various examples of braid patterns and braid intertwining techniques.

FIG. 8 is a partial cross-sectional side view of a portion of a delivery apparatus including the braid of FIG. 2.

FIG. 9 is a side elevation view of an exemplary embodiment of a braid including a looped portion.

FIG. 10 is a partial cross-sectional side view of a portion of a delivery apparatus including the braid of FIG. 9.

FIG. 11 is a side view of a bifurcated braid, according to one embodiment.

FIG. 12 is a cross-sectional view of a braid including a core member, according to one embodiment.

FIG. 13 is a side elevational view of a portion of another exemplary embodiment of a delivery apparatus.

### DETAILED DESCRIPTION

### General Considerations

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

All features described herein are independent of one another and, except where structurally impossible, can be used in combination with any other feature described herein.

As used in this application and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In the context of the present application, the terms “lower” and “upper” are used interchangeably with the terms “inflow” and “outflow”, respectively. Thus, for example, the lower end of the valve is its inflow end and the upper end of the valve is its outflow end.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device toward the user, while distal motion of the device is motion of the device away from the user. The terms “longitudinal” and “axial” refer to an axis extending in the proximal and distal directions, unless otherwise expressly defined.

### Examples of the Disclosed Technology

Described herein are prosthetic heart valves, delivery apparatus, and methods for implanting prosthetic heart valves. The disclosed delivery apparatus and methods can, for example, be used to implant a mechanically-expandable prosthetic heart valve, such as the valves described in U.S. Pat. No. 10,603,165 and U.S. Provisional Application No. 63/085,947, filed Sep. 30, 2020, each of which is incorporated herein by reference. For example, some mechanical valves can comprise pivotable junctions between the struts, while others can comprise a unitary lattice frame expandable and/or compressible via mechanical means. However, it should be appreciated that the delivery apparatuses described herein can additionally be used with other types of transcatheter prosthetic valves, including balloon-expandable prosthetic heart valves, such as disclosed in U.S. Pat. No. 9,393,110, and U.S. Publication Nos. U.S. 2018/0028310 and 2019/0365530, each of which are incorporated herein by reference, and self-expandable prosthetic heart valves, such as disclosed in U.S. Pat. No. 10,098,734, which is incorporated herein by reference.

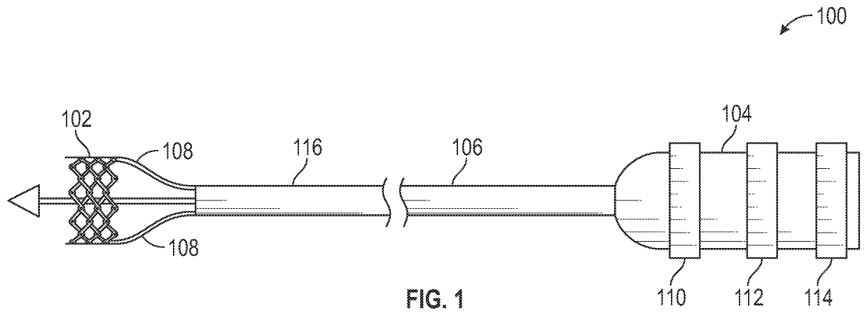

FIG. 1 illustrates an exemplary delivery apparatus 100 adapted to delivery a prosthetic heart valve 102. The prosthetic valve 102 can be releasably coupled to the delivery apparatus 100. Further, it should be understood that the delivery apparatus 100 and other embodiments of delivery apparatuses described herein can be used to implant prosthetic devices other than prosthetic valves, such as stents or grafts.

The delivery apparatus 100 in the illustrated embodiment generally includes a handle 104, a first elongated shaft 106 (which comprises an outer shaft in the illustrated embodiment) extending distally from the handle 104, at least one actuator assembly 108 extending distally through the outer shaft 106. The at least one actuator assembly 108 can be configured to radially expand and/or radially collapse the prosthetic valve 102 when actuated.

Though the illustrated embodiment shows two actuator assemblies 108 for purposes of illustration, it should be understood that one actuator 108 can be provided for each actuator on the prosthetic valve 102. For example, three actuator assemblies 108 can be provided for a prosthetic valve having three actuators. In other embodiments, a greater or fewer number of actuator assemblies can be present.

In some embodiments, a distal end portion 116 of the shaft 106 can be sized to house the prosthetic valve 102 in its radially compressed, delivery state during delivery of the prosthetic valve through the patient's vasculature. In this manner, the distal end portion 116 functions as a delivery sheath or capsule for the prosthetic valve during delivery,

The actuator assemblies 108 can be releasably coupled to the prosthetic valve 102. For example, in the illustrated embodiment, each actuator assembly 108 can be coupled to a respective actuator of the prosthetic valve 102. Each actuator assembly 108 can comprise a support tube, an inner actuator member (which can be, for example, a flexible tension member), and a locking tool. When actuated, the actuator assembly can transmit pushing and/or pulling forces to portions of the prosthetic valve to radially expand and collapse the prosthetic valve as previously described. The actuator assemblies 108 can be at least partially disposed radially within, and extend axially through, one or more lumens of the outer shaft 106. For example, the actuator assemblies 108 can extend through a central lumen of the shaft 106 or through separate respective lumens formed in the shaft 106.

The handle 104 of the delivery apparatus 100 can include one or more control mechanisms (e.g., knobs or other actuating mechanisms) for controlling different components of the delivery apparatus 100 in order to expand and/or deploy the prosthetic valve 102. For example, in the illustrated embodiment the handle 104 comprises first, second, and third knobs 110, 112, and 114.

The first knob 110 can be a rotatable knob configured to produce axial movement of the outer shaft 106 relative to the prosthetic valve 102 in the distal and/or proximal directions in order to deploy the prosthetic valve from the delivery sheath 116 once the prosthetic valve has been advanced to a location at or adjacent the desired implantation location with the patient's body. For example, rotation of the first knob 110 in a first direction (e.g., clockwise) can retract the sheath 116 proximally relative to the prosthetic valve 102 and rotation of the first knob 110 in a second direction (e.g., counter-clockwise) can advance the sheath 116 distally. In other embodiments, the first knob 110 can be actuated by sliding or moving the knob 110 axially, such as pulling and/or pushing the knob. In other embodiments, actuation of the first knob 110 (rotation or sliding movement of the knob 110) can produce axial movement of the actuator assemblies 108 (and therefore the prosthetic valve 102) relative to the delivery sheath 116 to advance the prosthetic valve distally from the sheath 116.

The second knob 112 can be a rotatable knob configured to produce radial expansion and/or contraction of the prosthetic valve 102. For example, rotation of the second knob 112 can move the actuator member and the support tube axially relative to one another. Rotation of the second knob 112 in a first direction (e.g., clockwise) can radially expand the prosthetic valve 102 and rotation of the second knob 112 in a second direction (e.g., counter-clockwise) can radially collapse the prosthetic valve 102. In other embodiments, the second knob 112 can be actuated by sliding or moving the knob 112 axially, such as pulling and/or pushing the knob.

The third knob 114 can be a rotatable knob configured to retain the prosthetic heart valve 102 in its expanded configuration. For example, the third knob 114 can be operatively connected to a proximal end portion of the locking tool of each actuator assembly 108. Rotation of the third knob 114 in a first direction (e.g., clockwise) can rotate each locking tool to resist radial compression of the frame of the prosthetic valve. Rotation of the knob 114 in the opposite direction (e.g., counterclockwise) can rotate each locking tool in the opposite direction to remove the locking tool from the respective inner actuator member. In other embodiments, the third knob 114 can be actuated by sliding or moving the third knob 114 axially, such as pulling and/or pushing the knob.

Although not shown, the handle 104 can include a fourth rotatable knob operative connected to a proximal end portion of each inner actuator member. The fourth knob can be configured to rotate each inner actuator member, upon rotation of the knob, to unscrew each actuator member from the proximal portion of the prosthetic valve 102. Once the locking tools and the actuator members are unscrewed from the prosthetic valve 102 they can be removed from the patient along with the support tubes.

In some embodiments, a delivery apparatus (e.g., delivery apparatus 100) can include one or more flexible members or sutures used to, for example, couple various components of the delivery apparatus to one another. For example, in some embodiments, components within the handle 104 of the delivery apparatus 100 can be coupled together using a flexible member and in other embodiments components such as the inner actuator members can comprise a flexible member. Depending on the nature of the components, it can be advantageous to vary the stiffness, tensile strength, diameter, length, and/or elongation of the flexible member to meet the demands of the system. In particular applications, it may be necessary for the flexible member to have a selected stiffness and a selected diameter. For example, the flexible member may have a high stiffness and a relatively small diameter while having a strength able to withstand an applied force of at least 120 N. In some embodiments, the flexible member may be able to withstand an applied force of up to 300 N.

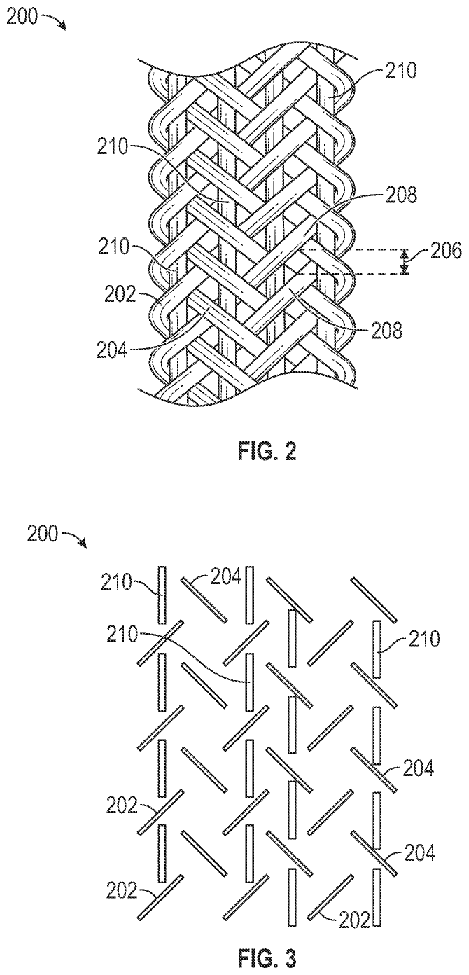

To provide the necessary strength and stiffness, in some embodiments, the flexible member or suture can be configured as a braid comprising a plurality of yarns. The yarns can comprise high tenacity and/or ultra-high strength materials. The yarns can be, for example, multi-filament (e.g., each yarn comprises a plurality of filaments) or mono-filament yarns (e.g., each yarn comprises a single filament) and can be intertwined by braiding into any of a variety of patterns, as described in more detail below with reference to FIGS. 2-7B.

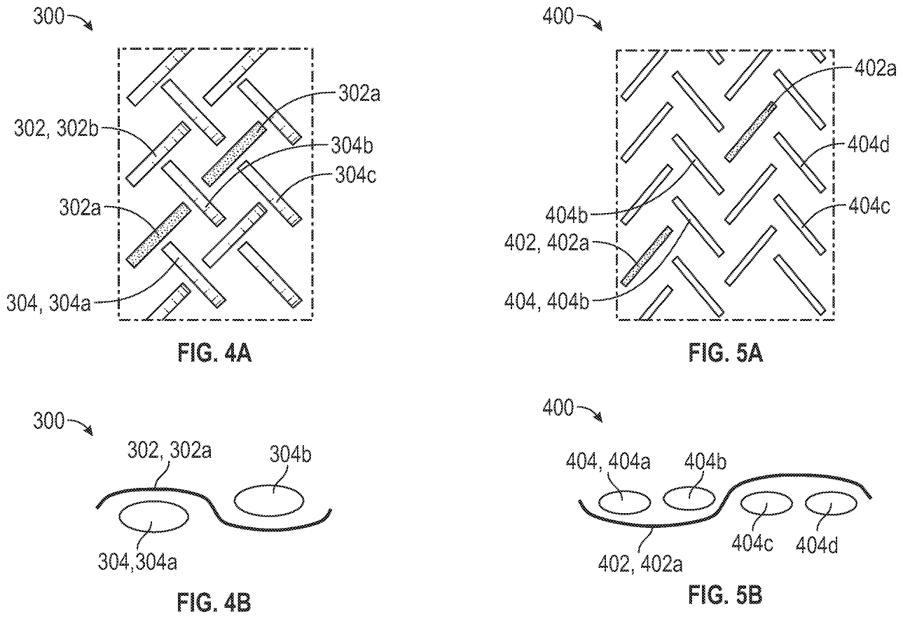

FIGS. 2-3 illustrate an exemplary braid 200 configured as a “plain braid” including one or more axially-extending yarns. As shown in FIGS. 4A-4B, a plain braid configuration 300 (also referred to as a ‘one yarn one-over, one-under braid’), includes first and second sets of yarns 302, 304 intertwined in a pattern in which a first yarn 302a passes over a second yarn 304a and then under a next second yarn 304b in a repeating pattern.

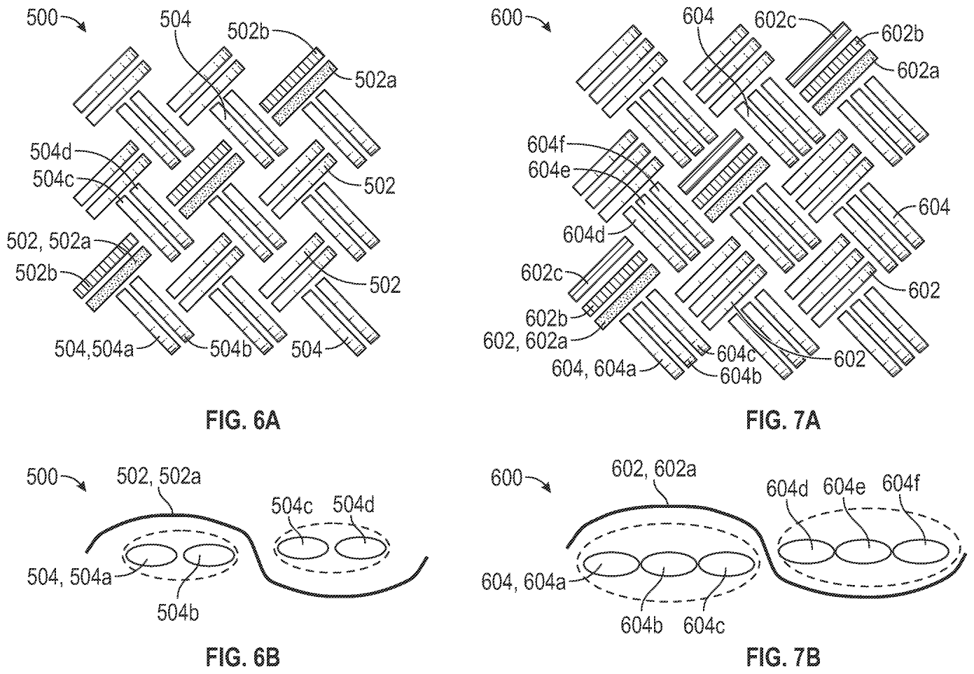

In other embodiments, the braid can have any of various braided configurations. For example, FIGS. 5A-5B illustrate a “regular braid” configuration 400 (also referred to as a ‘one yarn two-over, two-under’ pattern) having first and second sets of yarns 402, 404, in which a first yarn 402a passes under two second yarns 404a, 404b and then over a next set of two second yarns 404c, 404d in a repeating pattern. FIGS. 6A-6B illustrate a “diamond braid” configuration 500 (also referred to as a ‘two yarn two-over, two-under’ pattern) having first and second sets of yarns 502, 504 and in which two first yarns 502a, 502b pass over two second yarns 504a, 504b and then under a next set of two second yarns 504c, 504d in a repeating pattern. FIGS. 7A-7B illustrate a “Hercules braid” configuration 600 (also referred to as a ‘three yarn three-over, three-under’ pattern) having first and second sets of yarns 602, 604 in which three first yarns 602a, 602b, 602c pass over a first set of three second yarns 604a, 604b, 604c and the over a second set of three second yarns 604d, 604e, 604f in a repeating pattern.

Referring again to FIGS. 2-3, the plain braid can be configured as a tubular braid 200. Similarly, any of the braided configurations of FIGS. 4A-7B can be configured as a tubular braid. The braid can comprise a multi-filament yarn construction including a first set of yarns 202 and a second set of yarns 204. The first and second sets of yarns 202, 204 can comprise multi-filament yarns or mono-filament yarns. The first set of yarns 202 can spiral and intertwine in a first direction (e.g., clockwise) and the second set of yarns 204 can spiral and intertwine in a second direction (e.g., counter clockwise). The first and second sets of yarns 202, 204 can intertwine in a circular path to form a tubular braid. FIG. 3 illustrates a flattened view of the tubular braid 200 including axial yarns 210.

The stiffness of the braid 200 can be determined depending on the following braid parameters: braid density, type of yarn, yarn size (e.g., linear density), number of yarn ends in a braid, braid pattern and/or arrangement of braid structure (e.g., tubular braid), axial yarns, core members, and/or any combination thereof. The braid parameters can be varied to provide a braid with selected characteristics. Various braid parameters, dimensions, and other characteristics are described below in connection with a plain braid 200. However, it should be noted that the following disclosure of braid parameters, dimensions, and other characteristics can be used to form a braided member having any of the braid configurations of FIGS. 4A-7B or any other known braid configurations. Moreover, where plain braid 200 is shown or described herein as a component of a delivery apparatus, a braided member that is a component of a delivery apparatus can comprise any of the braid configurations disclosed herein.

As mentioned, the braid 200 can have a selected braid density that determines, at least in part, the stiffness of the braid. The braid density can be determined using a measurement of picks per inch (PPI). A pick 206, as shown in FIG. 2, is the area between two adjacent yarn crossover points 208. The PPI of a braid is the number of picks per inch along the longitudinal axis of the braid 200. A higher PPI results in a stiffer braid, while a lower PPI results in a more pliable braid. In some embodiments, the braid 200 can have a braid density of from about 10 PPI to about 400 PPI, from about 25 PPI to about 300 PPI, from about 50 PPI to about 200 PPI, from about 75 PPI to about 150 PPI, etc. In some particular embodiments, a selected braid density of 75 PPI was found to result in a desired stiffness for the braid 200. In particular embodiments, the stiffness of the braid 200 must be sufficient for the braid to pass through a small hole without the end portions of the braid 200 unravelling. That is, without the yarns 202, 204 of the braid 200 becoming separated from one another.

In some embodiments, it is desirable to form a braid having a selected diameter of less than 0.055 in. For example, in some embodiments, the diameter of the braid 200 can be between about 0.024 in and about 0.055 in. The number of yarns selected to form the braid 200 can vary depending on the size of the yarns and/or the selected diameter of the braid. In some embodiments, the tubular braid can comprise between 4 and 72 yarns. The linear density of the yarns (e.g., the measure of the yarn's mass per unit length) can be, for example, between 10 dtex to 500 dtex. Braids using yarns having a lower linear density can comprise a greater number of yarns, and braids using yarns having a higher linear density can comprise a fewer number of yarns. For example, a braid can comprise a first set of 32 yarns and a second set of 32 yarns, each yarn having a linear density of 25 dtex.

For braids wherein the desired diameter is small (e.g., less than 0.055 in), selecting a yarn with a smaller linear density, such as 110 dtex, allows for a braid having a relatively high number of yarns and selecting a yarn with a larger linear density, such as 440 dtex, allows for a braid having a relatively lower number of yarns. For example, in a particular embodiment, a tubular braid can comprise 16 yarns each having a linear density of 110 dtex. The 16 yarns can be separated into a first set of 8 yarns and a second set of 8 yarns and can be intertwined to form a braid having a diameter of less than 0.055 in. In another particular embodiment, a tubular braid can comprise 8 yarns each having a linear density of 440 dtex. The 8 yarns can be separated into a first set of 4 yarns and a second set of 4 yarns and can be intertwined to form a braid having a diameter of less than 0.055 in.

Depending on the strength requirements for the braid, the yarns can be formed from any of various materials. In some embodiments, the materials can be synthetic polymers with a tenacity of greater than 20 grams per denier (gpd). In other embodiments, the material can be selected from natural fibers (e.g., wool, silk, angora, cotton, flax, hemp, jute, etc.) and/or synthetic fibers (e.g., polypropylene, nylon, polyesters, polyethylene, aramids, polyaramids, liquid crystalline polymers, etc.). In some particular applications wherein the braid able to withstand an applied force of at least 120 N, the yarns can be ultra-high molecular weight polyethylene (UHMWPE). In some embodiments, the yarns can be biocompatible yarns. In some such embodiments, the biocompatible yarns can form a biocompatible braided member configured to be implanted within the body of a patient.

In some embodiments, the braid 200 can be formed using a “maypole” technique. Each yarn of the first and second sets of yarns 202, 204 can be coupled to a respective spool, and the spools can be intertwined over and under each other. Half of the spools (e.g., those coupled to the first set of yarns 202) can move in a first (e.g., clockwise) direction, and the other half of the spools (e.g., those coupled to the second set of yarns 204) can move in a second (e.g., counter clockwise) direction. Such configurations can produce a braid 200 having a relatively smooth outer surface. A smooth outer surface advantageously prevents or mitigates the braid 200 from catching and/or tearing on components (e.g., components of the delivery apparatus) when in use.

In some embodiments, the maypole technique can be performed using a carrier braider machine. The carrier braider machine can be configured to carry between 8 and 72 yarns having linear densities between about 10 dtex to about 500 dtex. In some particular embodiments, the carrier braider machine can carry 16 ends (configured as a first set of 8 yarns and a second set of 8 yarns) of yarn having a linear density of about 55 dtex. In other embodiments, the carrier braider machine can carry 64 ends (configured as a first set of 32 yarns and a second set of 32 yarns) of yarn having a linear density of about 25 dtex

In some embodiments, such as the embodiment illustrated in FIGS. 2-3, the braid 200 can comprise one or more axially-extending yarns 210 (also referred to as “axial yarns”). The axial yarns 210 can be configured to provide additional stiffness and/or strength to the braid 200. The axial yarns 210 can be, for example, multifilament yarns, monofilament yarns, and/or braided yarns. In some embodiments, the axial yarns 210 can be monofilament yarns having a diameter between about 0.001 inches and about 0.04 inches. In other embodiments, the axial yarns 210 can be multifilament yarns wherein the multifilament comprises, for example, 5 filaments each having a diameter of 0.001 inches.

The axial yarns 210 can be formed from any of various materials. In some embodiments, the materials can be synthetic polymers with a tenacity of greater than 20 grams per denier (gpd). In some embodiments, the materials can comprise natural fibers (e.g., wool, silk, angora, cotton, flax, hemp, jute, etc.) and/or synthetic fibers (e.g., polypropylene, nylon, polyesters, polyethylene, aramids, polyaramids, etc.). In some particular applications, the axial yarns 210 can comprise ultra-high molecular weight polyethylene (UHMWPE). In some other particular embodiments, the axial yarns can comprise liquid crystalline polymer (LCP). In some embodiments, the axial yarns can be biocompatible yarns.

As shown in FIG. 2, the axial yarns 210 can extend substantially parallel to a longitudinal axis of the braid 200. The axial yarns 210 can be interlaced with the first and/or second sets of yarns 202, 204 at selected locations, but are not part of the pattern of the braid 200. For example, the axial yarns 210 can be disposed between the first set of yarns 202 and the second set of yarns 204 (e.g., radially and/or along the circumference of the braided member). In the illustrated embodiment, the braid 200 has a plain braid configuration, however, axial yarns 210 can be incorporated in any of the above-described braid configurations (e.g., a regular braid, a diamond braid, a Hercules braid, etc.).

The braid 200 can comprise any number of axial yarns 210 depending on the strength requirements of the braid. For example, in the illustrated embodiment, the braid 200 comprises four axial yarns 210. Such a configuration can advantageously allow the braid to withstand an applied force of at least 120 N. In other embodiments, the braid 200 can comprise one, two, three, five, six, seven, eight, nine, or ten axial yarns. Though in the illustrated embodiment the four axial yarns 210 are shown on one side of the braid 200, in other embodiments the axial yarns 210 can be spaced apart from one another about the circumference of the braid 200. For example, in some embodiments, the four axial yarns 210 can be equally spaced and in other embodiments two or more axial yarns 210 can be disposed adjacent one another.

The axial yarns 210 can also prevent or mitigate elongation of the braid 200. When a force is applied to the braid 200 (e.g., a pulling force at a first and/or send end portion of the braid), the first and second sets of yarns 202, 204 pivot relative to one another to straighten relative to a longitudinal axis of the braid 200. The axial yarns 210 are disposed between the first and second sets of yarns 202, 204 such that they prevent the first and second sets of yarns 202, 204 from pivoting past a selected point and therefore from straightening. Such a configuration allows the braid 200 to be kept at a selected, finite length as required by the system.

Although not shown, any of the braid configurations of FIGS. 4A-7B can include axial yarns 210 incorporated into the braid as described above.

In some applications, it may be necessary to tie the braid 200 into a knot in order to retain the braid or a portion of the braid in a selected position (e.g., within a component). If a braid 200 does not have the selected stiffness required for a certain system, the knot may change size during pulling and/or twisting of the braid 200. The above embodiments describe braids wherein the braid density (PPI) of yarns (e.g., the tightness of the braid) prevents the knot from changing size during pulling and/or twisting. In one specific implementation, for example, a braid 200 comprises sixteen yarns of 110 dtex UHMWPE yarn, four axial yarns 210 of 110 dtex UHMWPE, intertwined at a PPI of 75.

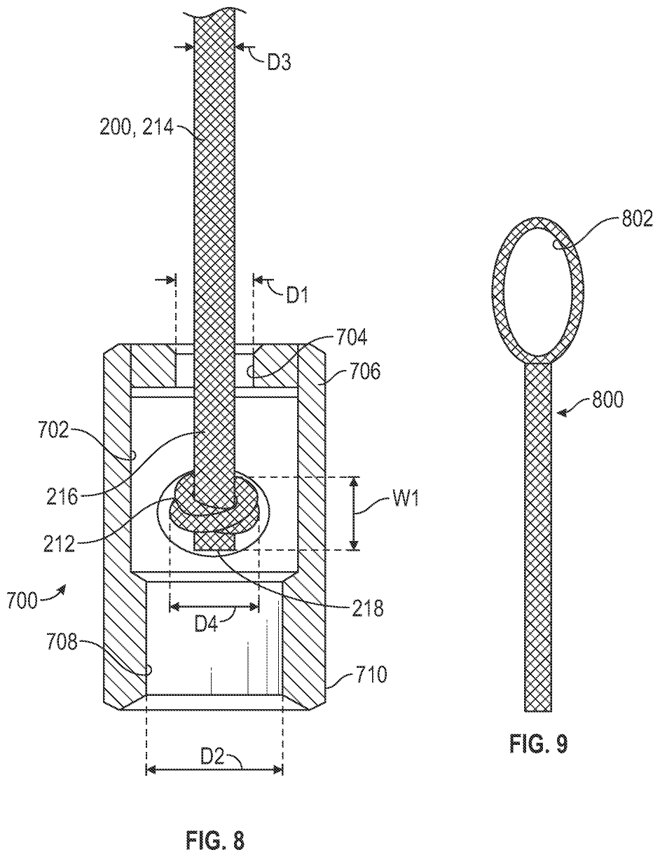

FIG. 8 illustrates an exemplary component of a delivery apparatus, namely, a cap member 700 of a force balancing assembly, such as force balancing assembly 1100 shown in FIG. 13. The cap member 700 can be a cylindrical member including an inner bore 702 and having a first aperture 704 having a first diameter D₁ at a first end portion 706 and a second aperture 708 having a second diameter D₂ at a second end portion 710. The first diameter D₁ can be narrower than the second diameter D₂. As shown in FIG. 8, a braid 200 can have an un-knotted diameter D₃ configured to allow the braid 200 to pass through the first aperture 704, and a knotted diameter D₄, which is greater than D₃ and D₁ and configured to restrain the braid 200 from passing through the first aperture 704. For example, in some particular embodiments, the first aperture 704 can have a diameter of 0.055 inches, the braid 200 can have a diameter D₃ between about 0.024 inches to about 0.055 inches, and the outer diameter D₄ of the knot 212 can be between about 0.060 inches and about 0.113 inches. In such embodiments, the braid 200 can comprise 16 yarns of 110 dtex UHMWPE, can include 4 axial yarns of 110 dtex UHMWPE, and can have a braided density of 75 PPI.

The knot 212 can further be configured to have a width Wi such that an end portion 218 of the knot 212 does not contact an actuation member (e.g., actuation member 1102 shown in FIG. 13) inserted into the second end portion 710 of the cap member 700. Contact between the knot 212 and an actuation member can cause twisting of the knot 212 and/or the braided member 200, which can result in a reduction in the length of the braided member 200 and thereby vary the tension. In some particular embodiments, the width Wi can be less than about 0.164 inches.

The knot 212 can couple the braid to the cap member 700 in the following exemplary manner. A first end portion 214 of the braid 200 can be inserted through the second aperture 708, through the inner bore 702, and out the first aperture 704. The first end portion 214 can continue to be threaded through the inner bore 702 until the knot 212 reaches the first aperture 704 at which point the diameter D₄ of the knot 212 prevents the knot 212 from passing through the first aperture 704, thereby retaining the knot 212 within the cap member 700 and coupling the braid 200 to the cap member. Alternatively, a second end portion of the braid 200 not yet including a knot 212 can be threaded through the first aperture 704 and into the inner bore 702. Once disposed within the inner bore 702, a portion of the second end portion 216 can be tied into the knot 212 to retain the braid 200 within the cap member 700.

Referring to FIG. 9, in some embodiments, in lieu of or in addition to a knot, the braid can be configured as a looped braid 800 having an end portion configured as a closed loop 802. The braid 800 can comprise any of the braid configuration disclosed herein, with the addition of the loop 802. The closed loop 802 can advantageously be formed on an end portion of the braid 800 such that there is no “free end” portion of the braid 800 that could potentially unravel. In some embodiments, the closed loop 802 can be formed by, for example, threading an end portion of the braid 800 through the eye of a needle, folding the needle back towards the braid, and piercing the needle into the braid 800 along the longitudinal axis of the braid for a selected distance. The needle can then extend out through a side of the braid 800 such that the end portion of the braid is at least partially trapped within the braid 800, causing the braid 800 to fold back on itself and form the closed loop 802. An end portion of the braid that extends out through the side of the braid 800 can be cut such that it is flush with the side of the braid, or knotted and/or adhered such that it cannot pass through the side of the braid. In other embodiments, the end portion of the braid can be folded back and glued and/or otherwise adhered to a side portion of the braid 800.

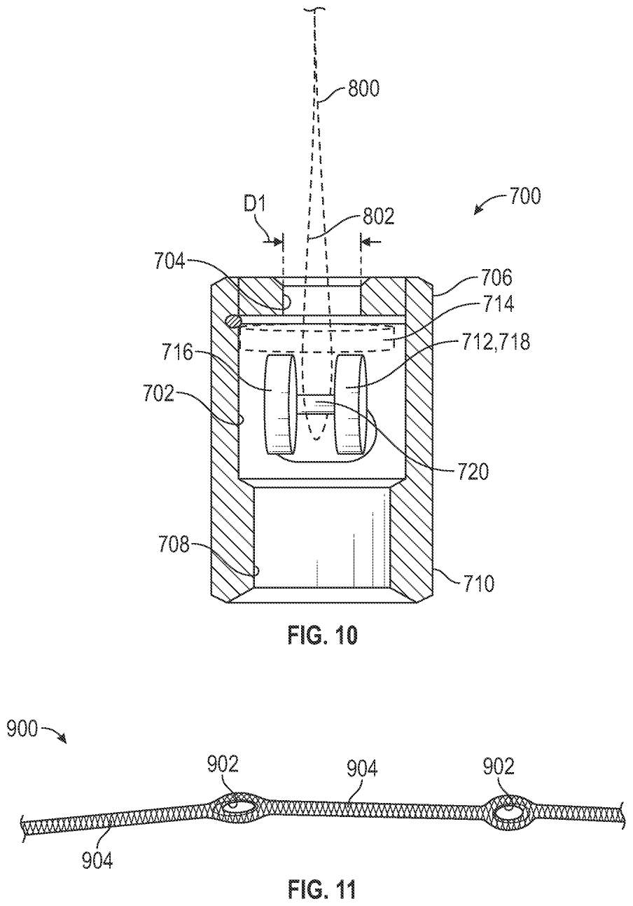

As shown in FIG. 10, the loop 802 can be used to secure the looped braid 800 to a cap member, such as cap member 700 described above, in the following exemplary manner. A retaining member 712 (e.g., an H-bracket) and a washer 714 can be disposed within the inner bore 702 of the cap member 700. The retaining member 712 can comprise first and second end portions 716, 718 separated by a neck portion 720 having a narrower diameter that the first and second end portions 716, 718. The retaining member 712 and/or the washer 714 can be sized such that they can not pass through the first aperture 704. The loop 802 of the looped braid 800 can be compressed (e.g., narrowing the loop opening) such that the loop 802 can pass through the first aperture 704 (and through an opening in the washer 714) and into the inner bore 702. Once disposed within the inner bore 702, the loop 802 can be allowed to re-expand (e.g., naturally or by applying a force to one or both ends of the loop 802) such that it can extend over the first or second end portion 716, 718 of the retaining member 712 and be disposed around the neck portion 720 as shown in FIG. 10. Such a method can advantageously secure the braid 800 to a component without the use of a knot, which can prevent or mitigate unravelling and/or knot slippage.

In still other embodiments, in lieu of or in addition to the knot and/or looped end portion, the braid can be configured as a bifurcated braid 900. As shown in FIG. 11, a bifurcated braid 900 can comprise one or more loop portions 902 between extending body portions 904 of the braid. The braid 900 can comprise any of the braid configuration disclosed herein, with the addition of the loop portions 902. In some embodiments, a bifurcated braid 900 can be coupled to a cap member, such as cap member 700, using the method described above for the looped braid 800. In such embodiments, the braided member 900 can be cut or severed on one side of a loop portion 902 such that the loop portion 902 can be inserted into a cap member such as cap member 700. In some embodiments, once the bifurcated braid 900 has been cut, the portion of the body 904 extending past the loop portion 902 can be tied into a knot (e.g., similar to knot 212) to prevent the cut portion from unravelling.

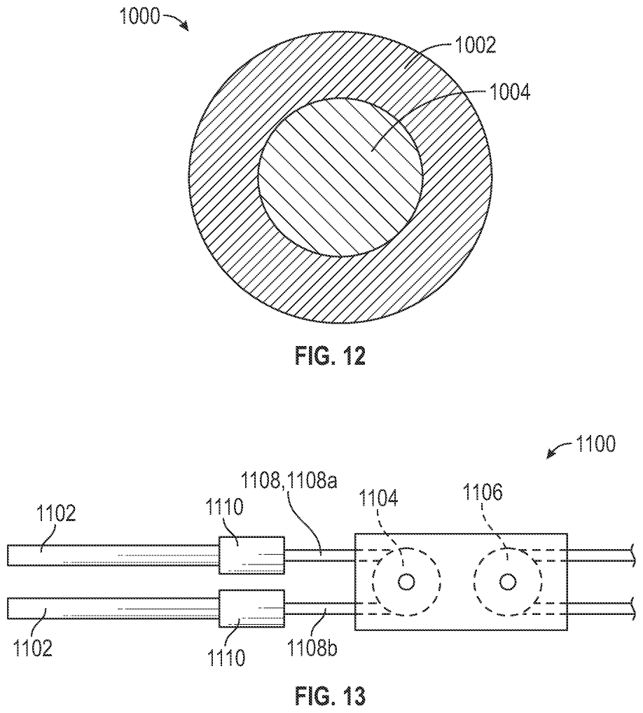

In some embodiments, as shown in FIG. 12, a braid 1000 can be configured as an outer braid 1002 disposed around an inner member or core 1004. The outer braid 1002 can comprise any of the above-described configurations (e.g., a plain braid with or without axial yarns, a regular braid, a diamond braid, etc.). The outer braid 1002 can be braided directly onto the core 1004. In some embodiments, the core 1004 can comprise a mono-filament yarn or a multi-filament yarn, and/or the core 1004 can be a wire or other member configured to add additional stiffness to the braid. In some embodiments, the core can comprise a mono-filament yarn having a diameter between about 0.001 inches and 0.04 inches. In other embodiments, the axial yarns 210 can be multifilament yarns wherein the multifilament comprises, for example, 5 filaments each having a diameter of 0.001 inches. Additional details of braids including core members can be found, for example, in U.S. Pat. No. 9,163,341, which is incorporated herein by reference in its entirety.

The above described braid members can be used in any of various locations within a delivery apparatus and/or prosthetic heart valve. For example, in some embodiments, a handle of a delivery apparatus (such as handle 104 described above) can include a tensioning or force balancing assembly 1100, a portion of which is shown in FIG. 13. The force balancing assembly 1100 can, for example, be configured to equally distribute the pulling force between two or more actuation members 1102 (similar to actuation members 108 described with respect to FIG. 1) using one or more balancing pulleys. Further details of the force balancing assembly and delivery apparatus can be found, for example, in International Application No. PCT/US2021/022467, which is incorporated herein by reference in its entirety.

The force balancing assembly 1100 can comprise a first pulley 1104 and a second pulley 1106. A flexible member configured as a braid 1108 (e.g., similar to braid 200 described previously), can extend around the first balancing pulley 1104. The first balancing pulley 1104 can rotate freely around its axis within the force balancing assembly 1100 in order to transfer tension between first and second end portions 1108a, 1108b of the braid 1108.

Each end portion 1108a, 1108b of the braid 1108 can be coupled to a respective actuation member 1102 via a respective cap member 1110 similar to cap member 700 described previously. Each cap member 1110 can have a diameter larger than a diameter of the actuation member 1102. As mentioned previously, the cap member 1110 can include an aperture or opening through which the braid 1108 can pass in order for the braid to be coupled to the cap member 1110. The braid 1108 can have a diameter selected such that the braid can pass through the opening, a selected stiffness such that a user can thread the braid through the opening without the braid 1108 unraveling, and a selected knot diameter such that when the braid 1108 is tied into a knot (see e.g., knot 212) the knot will restrain the braid from passing through the opening. For example, in a particular embodiment, the opening can have a diameter of 0.055 inches. In such embodiments, the braid 1108 can have a diameter between about 0.024 inches and about 0.055 inches and can form a knot between about 0.060 inches and 0.113 inches. Such a braid can be, for example, formed from 16 yarns of 110 dtex UHMWPE, can include 4 axial yarns of 110 dtex UHWPE, and can have a braid density of 75 PPI.

In some embodiments, an exemplary delivery apparatus for use with an exemplary prosthetic valve (e.g., delivery apparatus 100 and prosthetic valve 102 described previously) can include one or more actuator assemblies (e.g., actuator assembly 108) coupled to a distal end of the prosthetic valve. Each actuator assembly can comprise an outer support sleeve or tube configured to abut an outflow end portion of the prosthetic valve and an inner tension member or tether configured to couple an inflow end portion of the prosthetic valve. The tethers can be actuated to apply a proximally-directed force to the inflow end of the prosthetic valve while the support sleeves restrain (or apply a distally-directed force to) the outflow end of the prosthetic valve in order to move the prosthetic valve from a compressed configuration to an expanded configuration. In some embodiments, the tension members or tethers can be configured as braided members having any of the braid configurations described previously. Further details of the delivery apparatus, prosthetic valve, and actuator assemblies can be found, for example, in U.S. Pat. No. 10,603,165 and International Application Nos. PCT/US2020/057691 and PCT/US2020/063104 which are incorporated herein by reference in their entirety.

In some embodiments, a delivery apparatus (e.g., delivery apparatus 100) can further comprise a recapture device and/or crimping mechanism configured to facilitate crimping of a prosthetic valve, after the prosthetic valve has been exposed from the delivery apparatus inside the patient. The crimping mechanism can comprise a tension member formed as a loop and configured to extend around a portion of the delivery apparatus (e.g., the actuators 108) and/or the prosthetic valve (e.g., prosthetic valve 102). The tension member can be configured as a braided member having any of the configurations described previously. Further details of the crimping mechanism can be found, for example, in U.S. Publication No. 2020/0188099, which is incorporated herein by reference in its entirety.

The physician can deploy the distal end portion of the crimping mechanism from the delivery apparatus and then increase the size of the loop portion of the tension member (e.g., by applying a distally-directed force to the tension member). After increasing the size of the loop portion, the physician can move the tension member to slide the loop portion to a selected crimping location, such as around the circumference of the prosthetic valve. Once the tension member is in place around the prosthetic valve, the physician can contract the loop potion. This places the loop portion of the tension member in tension around the prosthetic valve which in turn applies a radially inwardly directed force to the prosthetic valve, thereby radially compressing the frame prosthetic valve.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

### Example 1

A braided member, comprising
a first set of yarns extending in a first direction;
a second set of yarns extending in a second direction and intertwined with the first set of yarns; and
a set of axial yarns extending along a longitudinal axis of the braided member and disposed between the first set of yarns and the second set of yarns;
wherein the braided member comprises a tubular braid.

### Example 2

The braided member of any example herein, particularly example 1, wherein a stiffness of the braided member can be selected based on one or more braid parameters, the braid parameters comprising braid density, yarn material, yarn linear density, number of yarns in the first and second sets, number of axial yarns, braid pattern, braid structure, and core members.

### Example 3

The braided member any example herein, particularly example 2, wherein the braid density is 10 PPI to 400 PPI.

### Example 4

The braided member of any example herein, particularly any one of examples 2-3, wherein the yarn material comprises at least one of ultra-high molecular weight polyethylene (UHMWPE) and liquid crystalline polymer.

### Example 5

The braided member of any example herein, particularly any one of examples 2-4, wherein the yarn linear density is 10 dtex to 500 dtex.

### Example 6

The braided member of any example herein, particularly any one of examples 2-5, wherein the first and second sets of yarns each comprise between 4 yarns and 32 yarns.

### Example 7

The braided member of any example herein, particularly any one of examples 2-6, wherein the braided member comprises at least 4 axial yarns.

### Example 8

The braided member of any example herein, particularly any one of examples 2-7, wherein the braid pattern comprises at least one of a plain braid, a regular braid, a diamond braid, and a Hercules braid.

### Example 9

The braided member of any of claims 2-8, wherein the braided member comprises a core member.

### Example 10

A braided member, comprising:
a first set of yarns extending in a first direction;
a second set of yarns extending in a second direction and intertwined with the first set of yarns;
a set of axial yarns extending along a longitudinal axis of the braided member and disposed between the first set of yarns and the second set of yarns; and
wherein the braided member has a braid density of between 10 PPI and 400 PPI.

### Example 11

The braided member of any example herein, particularly example 10, wherein the braided member has a braid density of 75 PPI.

### Example 12

The braided member of any example herein, particularly any one of examples 10-11, wherein the first set of yarns comprises 8 yarns each having a linear density of 110 dtex.

### Example 13

The braided member of any example herein, particularly any one of examples 10-12, wherein the second set of yarns comprises 8 yarns each having a linear density of 110 dtex.

### Example 14

The braided member of any example herein, particularly any one of examples 10-13, wherein the set of axial yarns comprises at least 4 yarns.

### Example 15

The braided member of any example herein, particularly any one of examples 10-11 or 14, wherein the first set of yarns comprises 4 yarns each having a linear density of 440 dtex.

### Example 16

The braided member of any example herein, particularly any one of examples 10-11, 14 or 15, wherein the second set of yarns comprises 4 yarns each having a linear density of 440 dtex.

### Example 17

The braided member of any example herein, particularly any one of examples 10-16, wherein each axial yarn has a linear density of 110 dtex.

### Example 18

The braided member of any example herein, particularly any one of examples 10-17, wherein each axial yarn has a linear density of 440 dtex.

### Example 19

The braided member of any example herein, particularly any one of examples 10-18, wherein the diameter of the braided member is less than 0.055 inches.

### Example 20

The braided member of any example herein, particularly any one of examples 10-19, wherein the outer diameter of a knot tied in the braid member is between 0.060 inches and 0.113 inches.

### Example 21

The braided member of any example herein, particularly any one of examples 10-20, wherein the braided member is configured to withstand a force of at least 120 N.

### Example 22

The braided member of any example herein, particularly any one of examples 10-21, wherein a first end portion of the braided member comprises a loop portion.

### Example 23

The braided member of any example herein, particularly any one of examples 10-22, further comprising one or more loop portions disposed along a longitudinal length of the braided member.

### Example 24

A braided member, comprising:
an outer layer comprising
   a first set of yarns extending in a first direction;
   a second set of yarns extending in a second direction and intertwined with the first set of yarns;
a set of axial yarns extending along a longitudinal axis of the braided member and disposed between the first set of yarns and the second set of yarns; and
an inner core member.

### Example 25

The braided member of any example herein, particularly example 24, wherein the core member is a monofilament yarn.

### Example 26

The braided member of any example herein, particularly example 24, wherein the core member is a multi-filament yarn.

### Example 27

The braided member of any example herein, particularly any one of examples 24-26, wherein the braided member has a braid density of 10 PPI to 400 PPI.

### Example 28

The braided member of any example herein, particularly any one of examples 24-27, wherein the braided member has a braid density of 75 PPI.

### Example 29

The braided member of any example herein, particularly any one of examples 24-27, wherein the first set of yarns comprises 8 yarns each having a linear density of 110 dtex.

### Example 30

The braided member of any example herein, particularly any one of examples 24-29, wherein the second set of yarns comprises 8 yarns each having a linear density of 110 dtex.

### Example 31

The braided member of any example herein, particularly any one of examples 24-30, wherein the set of axial yarns comprises at least 4 yarns.

### Example 32

The braided member of any example herein, particularly any one of examples 24-31, wherein each axial yarn has a linear density of 110 dtex.

### Example 33

The braided member of any example herein, particularly any one of examples 24-31, wherein each axial yarn has a linear density of 440 dtex.

### Example 34

The braided member of any example herein, particularly any one of examples 24-33, wherein the diameter of the braided member is less than 0.055 inches.

### Example 35

The braided member of any example herein, particularly any one of examples 24-34, wherein the outer diameter of a knot tied in the braid member is between 0.060 inches and 0.113 inches.

### Example 36

The braided member of any example herein, particularly any one of examples 24-35, wherein the braided member is configured to withstand a force of at least 120 N.

### Example 37

The braided member of any example herein, particularly any one of examples 24-36, wherein a first end portion of the braided member comprises a loop portion.

### Example 38

The braided member of any example herein, particularly any one of examples 24-37, further comprising one or more loop portions disposed along a longitudinal length of the braided member.

### Example 39

A delivery apparatus for implanting a prosthetic medical device comprising a braided member as disclosed in any example herein, particularly any one of examples 1-38.

### Example 40

The delivery apparatus of any example herein, particularly example 39, further comprising an actuator assembly configured to expand the prosthetic medical device, wherein the braided member is a component of the actuator assembly.

### Example 41

The delivery apparatus of any example herein, particularly example 40, further comprising a handle, two or more actuation members, and a force balancing assembly comprising the braided member coupling the actuation members to each other.

### Example 42

A force balancing assembly, comprising:
two or more actuation members each comprising a cap member;
at least one pulley member; and
at least one braided member having a first end portion coupled to a first cap member, a second end portion coupled to a second cap member, and a body portion disposed around the at least one pulley member, the braided member comprising
   a first set of yarns extending in a first direction,
   a second set of yarns extending in a second direction and intertwined with the first set of yarns,
a set of axial yarns extending along a longitudinal axis of the braided member and disposed between the first set of yarns and the second set of yarns;
wherein the force balancing assembly is configured to equally distribute a force between the two or more actuation members.

### Example 43

The force balancing assembly of any example herein, particularly example 42, wherein the braided member has a braid density between 10 and 400 PPI.

### Example 44

The force balancing assembly of any example herein, particularly example 42, wherein the cap member is a cylindrical member including an inner bore, and wherein the cap member has a first end portion including a first aperture.

### Example 45

The force balancing assembly of any example herein, particularly example 44, wherein the first aperture has a diameter of 0.055 inches or less.

### Example 46

The force balancing assembly of any example herein, particularly any one of examples 44-45, further comprising a retaining member and a washer disposed within the inner bore of the cap member.

### Example 47

The force balancing assembly of any example herein, particularly example 46, wherein the braided member further comprises a loop portion and wherein the loop portion extends through a central opening in the washer and is disposed around a portion of the retaining member.

### Example 48

The force balancing assembly of any example herein, particularly any one of examples 42-47, wherein the braided member has a braid density of 75 PPI.

### Example 49

The force balancing assembly of any example herein, particularly any one of examples 42-48, wherein the first and second sets of yarns each have a linear density of 110 dtex.

### Example 50

The force balancing assembly of any example herein, particularly any one of examples 42-49, wherein the set of axial yarns comprises at least 4 yarns.

### Example 51

The braided member of any example herein, particularly any one of examples 42-50, wherein each axial yarn has a linear density of 110 dtex.

### Example 52

The braided member of any example herein, particularly any one of examples 42-51, wherein the diameter of the braided member is less than 0.055 inches.

### Example 53

The braided member of any example herein, particularly any one of examples 42-52, wherein the outer diameter of a knot tied in the braid member is between 0.060 inches and 0.113 inches.

### Example 54

The braided member of any example herein, particularly any one of examples 42-53, further comprising one or more loop portions disposed along a longitudinal length of the braided member.

In view of the many possible embodiments to which the principles of the disclosure may be applied, it should be recognized that the illustrated embodiments are only preferred examples and should not be taken as limiting the scope of the disclosure. Rather, the scope is defined by the following claims. We therefore claim all that comes within the scope and spirit of these claims.

1. A braided member, comprising
a first set of yarns extending in a first direction;
a second set of yarns extending in a second direction and intertwined with the first set of yarns; and
a set of axial yarns extending along a longitudinal axis of the braided member and disposed between the first set of yarns and the second set of yarns;
wherein the braided member comprises a tubular braid.

2. The braided member of claim 1, wherein a stiffness of the braided member can be selected based on one or more braid parameters, the braid parameters comprising braid density, yarn material, yarn linear density, number of yarns in the first and second sets, number of axial yarns, braid pattern, braid structure, and core members.

3. The braided member of claim 2, wherein the braid density is 10 PPI to 400 PPI.

4. The braided member of claim 2, wherein the yarn material comprises at least one of ultra-high molecular weight polyethylene (UHMWPE) and liquid crystalline polymer.

5. The braided member of claim 2, wherein the yarn linear density is 10 dtex to 500 dtex.

6. The braided member of claim 2, wherein the first and second sets of yarns each comprise between 4 yarns and 32 yarns.

7. The braided member of claim 2, wherein the braided member comprises at least 4 axial yarns.

8. The braided member of claim 2, wherein the braid pattern comprises at least one of a plain braid, a regular braid, a diamond braid, and a Hercules braid.

9. The braided member of claim 2, wherein the braided member comprises a core member.

10. A braided member, comprising:
a first set of yarns extending in a first direction;
a second set of yarns extending in a second direction and intertwined with the first set of yarns;
a set of axial yarns extending along a longitudinal axis of the braided member and disposed between the first set of yarns and the second set of yarns; and
wherein the braided member has a braid density of between 10 PPI and 400 PPI.

11. The braided member of claim 10, wherein the braided member has a braid density of 75 PPI.

12. The braided member of claim 10, wherein the first set of yarns comprises 8 yarns each having a linear density of 110 dtex, or wherein the second set of yarns comprises 8 yarns each having a linear density of 11 dtex

13. The braided member of claim 10, wherein the set of axial yarns comprises at least 4 yarns.

14. The braided member of claim 10, wherein the first set of yarns comprises 4 yarns each having a linear density of 440 dtex, or wherein the second set of yarns comprises 4 yarns each having a linear density of 440 dtex.

15. The braided member of claim 10, wherein each axial yarn has a linear density between 110 dtex and 440 dtex.

16. The braided member of claim 10, wherein the diameter of the braided member is less than 0.055 inches.

17. The braided member of claim 10, wherein the outer diameter of a knot tied in the braid member is between 0.060 inches and 0.113 inches.

18. The braided member of claim 10, wherein the braided member is configured to withstand a force of at least 120 N.

19. The braided member of claim 10, wherein a first end portion of the braided member comprises a loop portion.

20. The braided member of claim 10, further comprising one or more loop portions disposed along a longitudinal length of the braided member.
